(19) United States
(12) Patent Application Publication
(10) Pub. No.: US 2009/0111780 A1
(43) Pub. Date: Apr. 30, 2009

(54) COMPOSITIONS AND METHODS FOR TREATMENT OF EAR CANAL INFECTION AND INFLAMMATION

(75) Inventor: John A Giordano, West Orange, NJ (US)

Correspondence Address: DON J. PELTO;Sheppard, Mullin, Richter & Hampton LLP, 1300 I STREET, NW, 11TH FLOOR EAST, WASHINGTON, DC, 20005, US

(73) Assignee: EVERETT LABORATORIES, INC.

(21) Appl. No.: 11/932,191

(22) Filed: Oct. 31, 2007

(51) Int. Cl. A61K 31/56; A61K 31/055; A61K 31/135; A61P 31/00

(52) U.S. Cl. 514/171; 514/737; 514/646

(57) ABSTRACT

The present invention relates to compositions and methods for treatment of ear infections and inflammation. Specifically, the method involves administering to a patient a composition containing an antibacterial, an anti-inflammatory, and an anti-septic that is substantially free of pramoxine to treat the occurrence or negative effects of otic infection and inflammation.

### FIELD OF THE INVENTION

The present invention relates to compositions comprising an antibacterial, an anti-inflammatory, and an anti-septic, and methods for administering these compositions for treatment of otic infections and inflammation.

### BACKGROUND OF THE INVENTION

The ear is susceptible to many disorders or infections. Ear infections can be categorized as either being an external ear infection, or a middle and inner ear infection. The middle ear consists of the eardrum (tympanic membrane) and a chamber that contains the ossicles that connect the eardrum to the inner ear. The eardrum in particular is susceptible to infection from viruses or bacteria which can cause severe pain. Such an infection, often called otitis media, is often induced by many factors, but it is most commonly due to a respiratory infection or water entering the middle ear.

The external ear consists of the auditory ear canal and the auricle. Otitis externa, sometimes called swimmer's ear or an external ear infection, is a commonly used term to define the infection or inflammation of the external auditory canal and auricle. Otitis externa may affect the entire ear canal, or just a small area within the canal. Otitis externa due to infection may be caused by a variety of bacteria or fungi such as the Staphylococcus species or the Corynebacterium species. Otitis externa may also be caused by inflammation such as from a scratch, from chemical irritants in hair spray or hair dye, or even from water. The Merck Manual of Medical Information (M. Beers et al., 2nd Home ed., Merck Research Laboratories, 2003). Another common cause of otitis externa is the use of earplugs or wearing hearing aids. In one study of 139 patients with otitis externa, 9% were found to have been wearing hearing aids (devices which couple to the ear with an earplug-like device). Hawke et al., 13 J. OTOLARYNGOL. 289-95 (1984). Because of the many causes, otitis externa is fairly prevalent, affecting about 3 to 5 percent of the population. Osguthorpe et al., 74 AMERICAN FAMILY PHYSICIAN Number 9 (2006).

Otitis externa can be classified in two forms: acute or chronic. Acute otitis externa is primarily of bacterial origin and is often associated with high humidity, warmer temperatures and swimming. Id. Alternatively, the chronic form is commonly caused from a fungal or allergic origin. Id. Although not caused by as many factors, chronic otitis externa is about ten times more prevalent than acute otitis externa. Id

Symptoms of external or middle ear infections are also variable due to the many causes. For example, otitis media symptoms may include pain due to a bulging eardrum or a discharge of pus. Common symptoms of otitis externa include itching and pain, and the ear canal may swell and contain pus or other form of discharge. M. Beers et al., supra.

Prevention and/or treatment of ear disorders or infections often come in the form of a suspension or solution. These preparations are usually placed topically in the ear canal by the administration of drops where the solution may sit in the ear canal for a designated amount of time. Ansel's Pharmaceutical Dosage Forms and Drug Delivery Systems (Ansel et al., 8th Edition, Lippincott Williams & Wilkins, 2005).

There are numerous drugs or medications that may be included in the ear drop solution. Many medications included in one solution are preferential due to the many ways that a patient can acquire otitis externa such as from bacteria or fungi. Hence, it would be useful to include medications such as an antibacterial, an anti-inflammatory, and an anti-septic all in one dosage form to treat the various causes of ear infection and inflammation such as otitis externa.

Benzalkonium chloride is a quaternary ammonium compound that has many uses. First, it may be used as an anti-septic and may be found in many first-aid sprays and lotions. Masson et al., 10 CURR. MED. CHEM. 1129-36 (2003). It may also be used as a preservative in products such as intranasal medicants. Benzalkonium chloride may also be used topically due to its effect on lowering surface tension, permitting better penetration of the skin of active compounds.

Hydrocortisone, also known as cortisol, is a glucocorticoid steroid that may be used to treat allergies and inflammation. Because swelling and inflammation are common symptoms of otitis externa and other ear infections, hydrocortisone may be a useful component for such treatment.

Chloroxylenol (parachlorometaxylenol) is a not toxic, non corrosive phenol. It is an antibacterial and is particularly effective against streptococcus bacteria. Berthelot et al., 17 DERMATITIS 156-9 (2006). Chloroxylenol is often used in antibacterial soaps. Its mechanism of action is by disruption of the of cell membrane potentials of bacterial organisms. Because of its

effective antibacterial activity, chloroxylenol may be an effective component for treatment of bacterial infections often associated with infections in the ear.

Many ear drop formulations contain a topical anesthetic for immediate relief of pain due to the infection or inflammation. For example, pramoxine is a nonsteroidal antipruritic that functions as a topical anesthetic. Topical anesthetics are commonly added in ear infection treatment compositions due to its ability to numb the pain due to ailments such as otitis externa. However, there are advantages to not including a topical anesthetic such as pramoxine in the composition. First, numbing the pain may mask symptoms of an advancing infection. Osguthorpe et al., supra. Hence, a topical anesthetic may hide symptoms that may cause serious if not permanent damage and be a risk that outweighs the quick relief it provides. Moreover, pramoxine is known to cause contact dermititis or induce inflammation to an allergic response. Adding pramoxine or another topical anesthetic may therefore knock out or reduce the benefits of hydrocortisone or other added anti-inflammatory within the composition.

### SUMMARY OF THE INVENTION

The present invention provides compositions and methods of using the compositions for otic use. Specifically, the present invention comprises a composition and methods of using an antibacterial, an anti-inflammatory, an anti-septic, and is substantially free of pramoxine.

In another embodiment of the present invention, the compositions may be substantially free of an anesthetic.

In another embodiment of the present invention, the compositions may comprise an antibacterial from one or more of the group consisting of chloroxylenol (parachlorometaxylenol), acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin and zorbamycin.

In another embodiment of the present invention, the compositions may comprise an anti-inflammatory from one or more of the group consisting of hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone prostaglandin H synthetase inhibitors (Cox I or Cox II), also referred to as cyclooxygenase type I and type II inhibitors, such as diclofenac, flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam and carprofen; cyclooxygenase type II selective inhibitors, such as NS-398, vioxx, celecoxib, P54, etodolac, L-804600 and S-33516; PAF antagonists, such as SR-27417, A-137491, ABT-299, apafant, bepafant, minopafant, E-6123, BN-50727, nupafant and modipafant; PDE IV inhibitors, such as ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast; inhibitors of cytokine production, such as inhibitors of the NFkB transcription factor.

In another embodiment of the present invention, the compositions may comprise an anti-septic from one or more of the group consisting of benzalkonium chloride, cetyl trimethylammonium bromide, cetylpyridinium chloride, cetylpyridinium chloride, benzethonium chloride, 2% aqueous iodine, boric acid, chlorhexidine gluconate, hydrogen peroxide, octenidine dihydrochloride, ethanol, isopropanol, n-propanol, phenylethyl alcohol, benzyl alcohol, and phenoxyethyl alcohol.

In one embodiment of the present invention, the compositions may comprise chloroxylenol (parachlorometaxylenol), benzalkonium chloride and hydrocortisone, where the compositions are administrable to a patient.

In another embodiment of the present invention, the compositions may be substantially free of other added active ingredients. The other added active ingredient may comprise another antibacterial such as one or more of the group consisting of acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefinenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin and zorbamycin. The other added active ingredient may comprise another anti-inflammatory such as one or more of the group consisting of hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone prostaglandin H synthetase inhibitors (Cox I or Cox II), also referred to as cyclooxygenase type I and type II inhibitors, such as diclofenac, flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam and carprofen; cyclooxygenase type II selective inhibitors, such as NS-398, vioxx, celecoxib, P54, etodolac, L-804600 and S-33516; PAF antagonists, such as SR-27417, A-137491, ABT-299, apafant, bepafant, minopafant, E-6123, BN-50727, nupafant and modipafant; PDE IV inhibitors, such as ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast; inhibitors of cytokine production, such as inhibitors of the NFkB transcription factor. The other added active ingredient may comprise another anti-septic such as one or more of the group consisting of cetyl trimethylammonium bromide, cetylpyridinium chloride, cetylpyridinium chloride, benzethonium chloride, 2% aqueous iodine, boric acid, chlorhexidine gluconate, hydrogen peroxide, octenidine dihydrochloride, ethanol, isopropanol, n-propanol, phenylethyl alcohol, benzyl alcohol, and phenoxyethyl alcohol.

In another embodiment, the compositions of the present invention may comprise one or more of about 1.5 mg to about 0.5 mg of chloroxylenol per 1 ml of solution; about 15 mg to about 5 mg of hydrocortisone per 1 ml of solution; about 0.15 mg to about 0.05 mg of benzalkonium chloride per 1 ml of solution.

In another embodiment, the compositions of the present invention may comprise one or more of about 1.25 mg to about 0.75 mg of chloroxylenol per 1 ml of solution; about 12.5 mg to about 7.5 mg of hydrocortisone per 1 ml of solution; about 0.125 mg to about 0.075 mg of benzalkonium chloride per 1 ml of solution.

In another embodiment, the compositions of the present invention may comprise one or more of about 1.1 mg to about 0.9 mg of chloroxylenol per 1 ml of solution; about 11 mg to about 9 mg of hydrocortisone per 1 ml of solution; about 0.11 mg to about 0.09 mg of benzalkonium chloride per 1 ml of solution.

In another embodiment of the present invention, the compositions may comprise one or more of about 1 mg of chloroxylenol per 1 ml of solution; about 10 mg of hydrocortisone per 1 ml of solution; and about 0.1 mg of benzalkonium chloride per 1 ml of solution.

In another embodiment of the present invention, the compositions may comprise a composition formulated within a vehicle.

In another embodiment of the present invention, the compositions may comprise a vehicle which is aqueous, non-aqueous, oleaginous and viscous.

In another embodiment of the present invention, the compositions may comprise a vehicle from one or more of the group consisting of glycols, alcohols, ethanol, isopropanol, glycerol, sorbitol, 2-methoxy ethanol, mannitol, diethyl ether, dipropyl ether, methoxy polyoxyethylenes, polyoxyethylene glycerols, polyoxyethylene sorbitols, stearoyl diacetin glycerin, dehydrated glycerin, mineral oil, water, anhydrous glycerin, glycerol polyvinyl alcohol, polyvinyl pyrrolidone, methyl cellulose, hydroxy propyl methylcellulose, hydroxyethyl cellulose, carboxymethyl cellulose, hydroxy propyl cellulose sorbitol, xylitol, cetrimide alkylaryltrialkylammonium chloride, alkylaryltrimethylammonium chloride, amantanium bromide, benzethonium chloride, benzododecinium bromide, cetalkonium chloride, cethexonium bromide, cetrimonium bromine, and cetyldimethylethylammonium bromide.

In another embodiment of the present invention, the compositions may comprise a glycol from one or more of the group consisting of glycol (PEG) 300 or any other molecular weight of PEG, propylene glycol, propylene glycol diacetate, diethylene glycol, ethylene glycol, hexalene glycol and hexenyl glycol.

In another embodiment of the present invention, the compositions may comprise one or more of about 2 percent to 4 percent of propylene glycol.

In another embodiment of the present invention, the compositions may comprise one or more of about 3 percent of propylene glycol.

In another embodiment of the present invention, the compositions may comprise a skin permeation enhancer from one or more of the group consisting of ethanol, isopropanol, n-alkanols, limonene, terpenes, dioxolane, propylene glycol, ethylene glycol, glycerol, dimethylsulfoxide (DMSO), dimethylformamide, methyl dodecyl sulfoxide, dimethylacetamide, isopropyl myristate/palmitate, ethyl acetate, butyl acetate, methyl proprionate, capric/caprylic triglycerides; ketones, acetamides, triolein; sodium lauryl sulfate, alkanoic acids, caprylic acid, azone, oleyl alcohol and dialkylamino acetates.

In another embodiment of the present invention, the compositions may be administered to a patient to treat and/or alleviate the occurrence or negative effects of otic inflammation.

In another embodiment of the present invention, the compositions may be administered to a patient to treat and/or alleviate the occurrence or negative effects of otic infection.

In another embodiment of the present invention, the compositions may be administered to a patient to treat and/or alleviate the occurrence or negative effects of otitis externa.

In another embodiment of the present invention, the compositions may be administered to a patient to treat and/or alleviate the occurrence or negative effects of otitis externa from one or more of the group consisting of acute otitis externa and chronic otitis externa.

In another embodiment of the present invention, the compositions may be administered to a patient to treat and/or alleviate the occurrence or negative effects of otic infections from one or more of the group consisting of furunculosis, otomycosis, acute diffuse otitis externa, acute localised otitis externa, chronic otitis externa, herpes oticus, dermatoses, malignant otitis externa, perichondritis, acute otitis media, serous otitis media, chronic otitis media, and vestibular neruonitis.

In another embodiment of the present invention, the compositions may be contained within a delivery system comprising a solution in a container that permits the administration of drops.

In another embodiment of the present invention, the methods may utilize compositions that are substantially free of an anesthetic.

In another embodiment of the present invention, the methods may utilize an antibacterial from one or more of the group consisting of chloroxylenol (parachlorometaxylenol), acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin and zorbamycin.

In another embodiment of the present invention, the methods may utilize an anti-inflammatory from one or more of the group consisting of hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone prostaglandin H synthetase inhibitors (Cox I or Cox II), also referred to as cyclooxygenase type I and type II inhibitors, such as diclofenac, flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam and carprofen; cyclooxygenase type II selective inhibitors, such as NS-398, vioxx, celecoxib, P54, etodolac, L-804600 and S-33516; PAF antagonists, such as SR-27417, A-137491, ABT-299, apafant, bepafant, minopafant, E-6123, BN-50727, nupafant and modipafant; PDE IV inhibitors, such as ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast; inhibitors of cytokine production, such as inhibitors of the NFkB transcription factor.

In another embodiment of the present invention, the methods may utilize an anti-septic from one or more of the group consisting benzalkonium chloride, cetyl trimethylammonium bromide, cetylpyridinium chloride, cetylpyridinium chloride, benzethonium chloride, 2% aqueous iodine, boric acid, chlorhexidine gluconate, hydrogen peroxide, octenidine dihydrochloride, ethanol, isopropanol, n-propanol, phenylethyl alcohol, benzyl alcohol, and phenoxyethyl alcohol.

In one embodiment of the present invention, the methods may include administering a composition comprising chloroxylenol, benzalkonium chloride and hydrocortisone.

In another embodiment of the present invention, the methods may utilize compositions substantially free of other added active ingredients. The other added active ingredient may be another antibacterial such as one or more of the group consisting of acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefinenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafingin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin and zorbamycin. The other added active ingredient may comprise another anti-inflammatory such as one or more of the group consisting of hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone prostaglandin H synthetase inhibitors (Cox I or Cox II), also referred to as cyclooxygenase type I and type II inhibitors, such as diclofenac, flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam and carprofen; cyclooxygenase type II selective inhibitors, such as NS-398, vioxx, celecoxib, P54, etodolac, L-804600 and S-33516; PAF antagonists, such as SR-27417, A-137491, ABT-299, apafant, bepafant, minopafant, E-6123, BN-50727, nupafant and modipafant; PDE IV inhibitors, such as ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast; inhibitors of cytokine production, such as inhibitors of the NFkB transcription factor. The other added active ingredient may comprise another anti-septic such as one or more of the group consisting of cetyl trimethylammonium bromide, cetylpyridinium chloride, cetylpyridinium chloride, benzethonium chloride, 2% aqueous iodine, boric acid, chlorhexidine gluconate, hydrogen peroxide, octenidine dihydrochloride, ethanol, isopropanol, n-propanol, phenylethyl alcohol, benzyl alcohol, and phenoxyethyl alcohol.

In another embodiment of the present invention, the methods may utilize compositions comprising one or more of about 1.5 mg to about 0.5 mg of chloroxylenol; about 15 mg to about 5 mg of hydrocortisone; and about 0.15 mg to about 0.05 mg of benzalkonium chloride.

In another embodiment of the present invention, the methods may utilize compositions comprising one or more of about 1.5 mg to about 0.5 mg of chloroxylenol per 1 ml of solution; about 15 mg to about 5 mg of hydrocortisone per 1 ml of solution; about 0.15 mg to about 0.05 mg of benzalkonium chloride per 1 ml of solution.

In another embodiment of the present invention, the methods may utilize compositions comprising one or more of about 1.25 mg to about 0.75 mg of chloroxylenol per 1 ml of solution; about 12.5 mg to about 7.5 mg of hydrocortisone per 1 ml of solution; about 0.125 mg to about 0.075 mg of benzalkonium chloride per 1 ml of solution.

In another embodiment of the present invention, the methods may utilize compositions comprising one or more of about 1.1 mg to about 0.9 mg of chloroxylenol per 1 ml of solution; about 11 mg to about 9 mg of hydrocortisone per 1 ml of solution; about 0.11 mg to about 0.09 mg of benzalkonium chloride per 1 ml of solution.

In another embodiment of the present invention, the methods may utilize compositions comprising one or more of about 1 mg of chloroxylenol per 1 ml of solution; about 10 mg of hydrocortisone per 1 ml of solution; and about 0.1 mg of benzalkonium chloride per 1 ml of solution.

In another embodiment of the present invention, the methods may utilize compositions formulated in a vehicle.

In another embodiment of the present invention, the methods may utilize compositions comprising one or more of a vehicle which is aqueous, non-aqueous, oleaginous and viscous.

In another embodiment of the present invention, the methods may utilize compositions comprising a vehicle selected from one or more of the group consisting of glycols, alcohols, ethanol, isopropanol, glycerol, sorbitol, 2-methoxy ethanol, mannitol, diethyl ether, dipropyl ether, methoxy polyoxyethylenes, polyoxyethylene glycerols, polyoxyethylene sorbitols, stearoyl diacetin glycerin, dehydrated glycerin, mineral oil, water, anhydrous glycerin, glycerol polyvinyl alcohol, polyvinyl pyrrolidone, methyl cellulose, hydroxy propyl methyl cellulose, hydroxyethyl cellulose, carboxymethyl cellulose, hydroxy propyl cellulose sorbitol, xylitol, cetrimide alkylaryltrialkylammonium chloride, alkylaryltrimethylammonium chloride, amantanium bromide, benzethonium chloride, benzododecinium bromide, cetalkonium chloride, cethexonium bromide, cetrimonium bromine, and cetyldimethylethylammonium bromide.

In another embodiment of the present invention, the methods may utilize compositions comprising a glycol from one or more of the group consisting of polyethylene glycol (PEG) 300 or any other molecular weight of PEG, propylene glycol, propylene glycol diacetate, diethylene glycol, ethylene glycol, hexalene glycol and hexenyl glycol.

In another embodiment of the present invention, the methods may utilize a composition comprising one or more of about 2 percent to 4 percent of propylene glycol.

In another embodiment of the present invention, the methods may utilize a composition comprising one or more of about 3 percent of propylene glycol.

In another embodiment of the present invention, the methods may utilize a composition comprising a skin permeation enhancer from one or more of the group consisting of ethanol, isopropanol, n-alkanols, limonene, terpenes, dioxolane, propylene glycol, ethylene glycol, glycerol, dimethylsulfoxide (DMSO), dimethylformamide, methyl dodecyl sulfoxide, dimethylacetamide, isopropyl myristate/palmitate, ethyl acetate, butyl acetate, methyl proprionate, capric/caprylic triglycerides; ketones, acetamides, triolein; sodium lauryl sulfate, alkanoic acids, caprylic acid, azone, oleyl alcohol and dialkylamino acetates.

In another embodiment of the present invention, the methods may include administering to a patient to treat and/or alleviate the occurrence or negative effects of otic inflammation.

In another embodiment of the present invention, the methods may include administering to a patient to treat and/or alleviate the occurrence or negative effects of otic infection.

In another embodiment of the present invention, the methods may include administering to a patient to treat and/or alleviate the occurrence or negative effects of otitis externa.

In another embodiment of the present invention, the methods may include administering to a patient to treat and/or alleviate the occurrence or negative effects of otitis externa from one or more of the group consisting of acute otitis externa and chronic otitis externa.

In another embodiment of the present invention, the methods may include administering to a patient to treat and/or alleviate the occurrence or negative effects of otic infections from one or more of the group consisting of furunculosis, otomycosis, acute diffuse otitis externa, acute localised otitis externa, chronic otitis externa, herpes oticus, dermatoses, malignant otitis externa, perichondritis, acute otitis media, serous otitis media, chronic otitis media, and vestibular neruonitis.

In another embodiment of the present invention, the methods may utilize compositions contained within a delivery system consisting of a solution in a container that permits the administration of drops.

In another embodiment of the present invention, the methods may utilize compositions that may be administered to a patient at a frequency selected from the group consisting of once a day, twice a day, three times a day, four times a day, five times a day, six times a day, seven times a day, eight times a day, nine times a day, ten times a day, eleven times a day and twelve times a day.

In another embodiment of the present invention, the methods may utilize compositions that a patient takes at a frequency selected from the group consisting of once a day, twice a day, three times a day, four times a day, five times a day, six times a day, seven times a day, eight times a day, nine times a day, ten times a day, eleven times a day and twelve times a day.

In another embodiment of the present invention, the methods may utilize compositions that may be administered to a patient at a frequency selected from the group consisting of once every hour, once every two hours, once every three hours, once every four hours, once every five hours, once every six hours, once every seven hours, once every eight hours, once every nine hours, once every ten hours, once every eleven hours, once every twelve hours, once every thirteen hours, once every fourteen hours, once every fifteen hours, once every sixteen hours, once every seventeen hours, once every eighteen hours, once every nineteen hours, once every twenty hours, once every twenty one hours, once every twenty two hours, once every twenty three hours and once every twenty four hours.

In another embodiment of the present invention, the methods may utilize compositions that a patient takes at a frequency selected from the group consisting of once every hour, once every two hours, once every three hours, once every four hours, once every five hours, once every six hours, once every seven hours, once every eight hours, once every nine hours, once every ten hours, once every eleven hours, once every twelve hours, once every thirteen hours, once every fourteen hours, once every fifteen hours, once every sixteen hours, once every seventeen hours, once every eighteen hours, once every nineteen hours, once every twenty hours, once every twenty one hours, once every twenty two hours, once every twenty three hours and once every twenty four hours.

In another embodiment of the present invention, the methods may utilize compositions that may be administered to a patient at a frequency selected from the group consisting of 1 drop, 2 drops, 3 drops, 4 drops, 5 drops, 6 drops, 7 drops and 8 drops.

### DETAILED DESCRIPTION OF THE INVENTION

It is understood that the present invention is not limited to the particular methodologies, protocols, fillers, vehicles, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an anti-inflammatory” is a reference to one or more anti-inflammatories and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “patient,” as used herein, comprises any and all organisms and includes the term “subject.” “Patient” may refer to a human or any other animal, including mammals.

The term “administrable” defines a composition that is able to be given to a patient. Likewise, “administering” refers to the act of giving a composition to a patient or otherwise making such composition available to a patient or the patient taking a composition.

The term “active ingredient” as used herein is any ingredient that is an anti-septic, anti-inflammatory, and antibacterial.

As used herein, the terms “inactive,” “inert,” “excipient,” and/or “formulatory” refer to any compound that is an inactive ingredient of a described composition. The definition of “inactive ingredient” as used herein follows that of the U.S. Food and Drug Administration, as defined in 21 C.F.R. 201.3(b)(8), which is any component of a drug product other than the active ingredient. By “active ingredient,” then, is meant any compound intended to furnish pharmacological activity or other direct effect in the diagnosis, cure, mitigation, treatment and/or prevention of a condition. See 21 C.F.R. 210.3(b)(7). Further, “active ingredients” include those compounds of the composition that may undergo chemical change during the manufacture of the composition and be present in the final composition in a modified form intended to furnish an activity or effect. Id.

Herein, “antibacterial” is defined herein as a compound that destroys or inhibits the growth of bacteria and may be referred to as an antibiotic or bacteriostat.

Herein, “anti-septic” is defined as an antimicrobial that may be applied to the skin of the patient.

The term “substantially free,” as used herein, means free from therapeutically effective amounts of compounds when administered in suggested dosages, but may include trace amounts of compounds in non-therapeutically effective amounts.

The term “dosage form,” as used herein, is the form in which the dose is to be administered to the patient. The drug is generally administered as part of a formulation that includes non-medical agents, referred to as pharmaceutical ingredients.

The ear is susceptible to many disorders or infections. Ear infections can be categorized as either being an external ear infection, or a middle and inner ear infection.

Middle and inner ear disorders may include infectious myringitis, mastoiditis, acute otitis media, serous otitis media, chronic otitis media, Meniere's disease and vestibular neruonitis. The middle ear consists of the eardrum (tympanic membrane) and a chamber that contains the ossicles that connect the eardrum to the inner ear. The eardrum in particular is susceptible to infection from viruses or bacteria which can cause severe pain. Such an infection, often called otitis media, is often induced by many factors, but it is most commonly due to a respiratory infection or water entering the middle ear.

Otitis externa, sometimes called swimmer's ear or an external ear infection, is a common form of infection or inflammation of the external auditory canal and auricle. Otitis externa may affect the entire ear canal, or just a small area within the canal. Otitis externa from infection may be caused by a variety of bacteria or fungi such as the Staphylococcus species or the Corynebacterium species. Otitis externa may also be caused by inflammation such as from a scratch, from chemical irritants in hair spray or hair dye, or water. The Merck Manual of Medical Information (M. Beers et al., 2nd Home ed., Merck Research Laboratories, 2003). Another common cause of otitis externa is the use of earplugs or wearing hearing aids. In one study of 139 patients with otitis externa, 9% were found to have been wearing hearing aids (devices which couple to the ear with an earplug-like device). Hawke et al., 13 J. OTOLARYNGOL. 289-95 (1984). Because of the many causes, otitis externa is fairly prevalent, affecting about 3 to 5 percent of the population. Osguthorpe et al., 74 AMERICAN FAMILY PHYSICIAN Number 9 (2006).

Otitis externa can be classified in two forms: acute or chronic. Acute otitis externa is primarily of bacterial origin and is often associated with high humidity, warmer temperatures and swimming. Id. Alternatively, the chronic form is commonly caused from a fungal or allergic origin. Id. Although not caused by as many factors, chronic otitis externa is about ten times more prevalent than acute otitis externa. Id.

Symptoms of external or middle ear infections are also variable due to the many causes. For example, otitis media symptoms may include pain due to a bulging eardrum or a discharge of pus. Common symptoms of otitis externa include itching and pain, and the ear canal may swell and contain pus or other form of discharge. M. Beers et al., supra.

Prevention and/or treatment of ear disorders or infections often come in the form of a suspension or solution. These preparations are usually placed topically in the ear canal by the administration of drops where the solution may sit in the ear canal for a designated amount of time. Ansel's Pharmaceutical Dosage Forms and Drug Delivery Systems (Ansel et al., 8th Edition, Lippincott Williams & Wilkins, 2005).

There are numerous drugs or medications that may be included in the ear drop solution. Many medications included in one solution are preferential due to the many ways that a patient can acquire otitis externa such as bacteria or fungi. Hence, it would be useful to include medications such as an antibacterial, an anti-inflammatory, and an anti-septic all in one dosage form to treat the various causes of otitis externa.

In one specific embodiment, the compositions and methods may comprise an anti-septic such as benzalkonium chloride, cetyl trimethylammonium bromide, cetylpyridinium chloride, benzethonium chloride, 2% aqueous iodine, boric acid, chlorhexidine gluconate, hydrogen peroxide, octenidine dihydrochloride, ethanol, isopropanol, n-propanol, phenylethyl alcohol, benzyl alcohol, and phenoxyethyl alcohol. For example, cetylpyridinium chloride is a cationic quaternary ammonium compound that is commonly found in mouthwashes due its strong anti-septic properties. Gunsolley, 137 J. AM. DENT. ASSOC. 1649-57 (2006).

Benzalkonium chloride is also a cationic quaternary ammonium compound that may be used as an anti-septic in various applications. For example, benzalkonium chloride may be used as an antiseptic in many first-aid sprays and lotions. Masson et al., 10 CURR. MED. CHEM. 1129-36 (2003). Benzalkonium chloride may also be effective when applied topically to the patient due to its effect on lowering surface tension. Hence, benzalkonium chloride is useful for permitting better penetration of the skin of active compounds. Benzalkonium chloride may also be used as a preservative, such as in intranasal products. Marple et al., 130 OTOLARYNGOL. HEAD NECK SURG. 131-41 (2004).

In one specific embodiment, the compositions and methods may comprise benzalkonium chloride. Specifically, the amounts may range from about 0.15 mg to about 0.05 mg per 1 ml of solution. In another specific embodiment of the present invention, the compositions and methods may comprise benzalkonium chloride in amounts ranging from about 0.125 mg to about 0.075 mg per 1 ml of solution. In another specific embodiment of the present invention, the compositions and methods may comprise benzalkonium chloride in amounts ranging from about 0.11 mg to about 0.09 mg per 1 ml of solution. In another specific embodiment of the present invention, the compositions and methods may comprise benzalkonium chloride in an amount of about 0.1 mg per 1 ml of solution.

In another embodiment, the compositions and methods may comprise an anti-inflammatory such as hydrocortisone (cortisol), hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone prostaglandin H synthetase inhibitors (Cox I or Cox II), also referred to as cyclooxygenase type I and type II inhibitors, such as diclofenac, flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam and carprofen; cyclooxygenase type II selective inhibitors, such as NS-398, vioxx, celecoxib, P54, etodolac, L-804600 and S-33516; PAF antagonists, such as SR-27417, A-137491, ABT-299, apafant, bepafant, minopafant, E-6123, BN-50727, nupafant and modipafant; PDE IV inhibitors, such as ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast; inhibitors of cytokine production, such as inhibitors of the NFkB transcription factor. For example, ibuprofen inhibits the role of COX-2 to induce prostaglandin synthesis and hence, primarily inhibits the inflammatory response. Smith et al., 69 ANNU. REV. BIOCHEM. 145-82 (2000).

Hydrocortisone, also known as cortisol, is an anti-inflammatory agent that can be used to treat allergies and inflammation. Specifically, hydrocortisone is a member of the glucocorticoid steroid hormone family. Glucocorticoids perform their function by binding to its receptor, the glucocorticoid receptor in the cytoplasm. Schacke et al., 15 EXP. DERMATOL. 565-73 (2006). This binding activates the receptor, which then subsequently transports into the nucleus and regulates the expression of a diverse array of proteins as a transcription factor. Id. For example, it is believed that the glucocorticoid receptor down regulates pro immunogenic response proteins such as IL-2. Id. This would account for the steroidal hormone's anti-inflammatory properties and therefore, hydrocortisone may be useful for treatment against swelling and inflammation that accompany ear infections.

In one specific embodiment, the compositions and methods may comprise hydrocortisone. Specifically, the amounts may range from about 15 mg to about 5 mg per 1 ml of solution. In another specific embodiment of the present invention, the compositions and methods may comprise hydrocortisone in amounts ranging from about 12.5 mg to about 7.5 mg per 1 ml of solution. In another specific embodiment of the present invention, the compositions and methods may comprise hydrocortisone in amounts ranging from about II mg to about 9 mg per 1 ml of solution. In another specific embodiment of the present invention, the compositions and methods may comprise hydrocortisone in an amount of about 10 mg per 1 ml of solution.

In another embodiment, the compositions and methods may comprise an antibacterial or other antibiotic agent selected from the group consisting of chloramphenicol, tetracyclines, synthetic and semi-synthesic penicillins, beta-lactames, quinolones, fluoroquinolnes, macrolide antibiotics, peptide antibiotics, cyclosporines. Specifically these antibacterial agents may include chloroxylenol (parachlorometaxylenol), acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; zorbamycin; and combinations thereof.

Chloroxylenol is an antibacterial that may be used in commercial antibacterial soaps and is particularly effective against streptococcus bacteria. Berthelot et al., 17 DERMATITIS 156-9 (2006). Chloroxylenol's mechanism of action works by disrupting the cell membrane potentials of bacterial organisms. Because of its effective antibacterial activity, chloroxylenol may be an effective component for treatment of bacterial infections often associated with ear infections and otitis externa.

In one specific embodiment, the compositions and methods may comprise chloroxylenol. Specifically, the amounts may range from about 1.5 mg to about 0.5 mg per 1 ml of solution. In another specific embodiment of the present invention, the compositions and methods may comprise chloroxylenol in amounts ranging from about 1.25 mg to about 0.75 mg per 1 ml of solution. In another specific embodiment of the present invention, the compositions and methods may comprise chloroxylenol in amounts ranging from about 1.1 mg to about 0.9 mg per 1 ml of solution. In another specific embodiment of the present invention, the compositions and methods may comprise chloroxylenol in an amount of about 1 mg per 1 ml of solution.

In another specific embodiment, the methods and compositions of the present invention may be free of other added active ingredients. The addition of other active ingredients can produce adverse side effects that can inhibit or outweigh the benefits of the compositions of the present invention. For example, isopropanol may be added as an anti-septic. However, isopropanol can sting and it evaporates quickly, which may afford a dry ear canal that may cause irritation. Osguthorpe et al., supra. In a specific embodiment, the compositions and methods of the present invention may be free of isopropanol.

Non-steroidal anti-inflammatory drugs (NSAIDs) such as aspirin are widely used for their analgesic and anti-inflammatory effects. However, current evidence suggests that adverse side effects, such as gastrointestinal and other bleeding risks of NSAIDs, probably outweigh its potential benefits. Kim et al., 58 J. PHARM. PHARMACOL. 1295-1304 (2006). In a specific embodiment, the compositions and methods of the present invention may be free of non-steroidal anti-inflammatory drugs such as aspirin.

Neomycin is an aminoglycoside antibacterial that can be used in ointments and creams. However, neomycin may induce a hypersensitive reaction from the patient and can incite contact dermatitis within the ear canal. Osguthorpe et al., supra. In a specific embodiment, the compositions and methods of the present invention may be free of antibacterials such as neomycin.

Prolonged use of the anti-inflammatory desonide has adverse symptoms such as causing redness, blistering, burning, itching, or peeling of the skin. In a specific embodiment, the composition and methods of the present invention may be free of anti-inflammatories such as desonide.

Herein, the term “vehicle” is intended to mean any compound that may be used as a carrier of other compounds or ingredients, any compound that may used as a solvent, any compound that is used to solubilize active ingredients, any compound that is used to increase the viscosity of the solution, or any compound used in any manner where the vehicle may be an aqueous, non-aqueous or oleaginous solution.

Exemplary vehicles include both monohydric and polyhydric alcohols, for example ethanol, isopropanol, glycerol, sorbitol, 2-methoxy ethanol, diethylene glycol, ethylene glycol, hexalene glycol, mannitol, and propylene glycol; ethers, such as diethyl or dipropyl ether; polyethylene glycols and methoxy polyoxyethylenes (such as carbowaxes having molecular weights ranging from 200 to 20,000); polyoxyethylene glycerols, polyoxyethylene sorbitols, and stearoyl diacetin.

The vehicle may be glycerin, a glycol, dehydrated glycerin, mineral oil, water, anhydrous glycerin, glycerol polyvinyl alcohol, polyvinyl pyrrolidone, methyl cellulose, hydroxy propyl methylcellulose, hydroxyethyl cellulose, carboxymethyl cellulose, hydroxy propyl cellulose sorbitol and xylitol.

The glycol may be of polyethylene glycol 300 (PEG) or any other form or molecular weight of PEG, propylene glycol, polyethylene glycol and ethylene glycol.

The vehicle may be the propellant or a mixture of the propellant and co-solvents such as alcohol, propylene glycol, and polyethylene glycols which are often used to enhance the solubility of the active ingredients. The amount of the vehicle may be balanced to solubilize the medicament. These vehicles may be used to increase solubility of compounds in a water solution. For example, hydrocortisone is nearly insoluble in water (0.028% on a weight volume basis).

Therefore, due to the low solubility of hydrocortisone in many aqueous solvents, co-solvents such as lower molecular weight alcohols, such as ethanol and glycols (propylene glycol, hexenyl glycol) may be added to increase the solubility of hydrocortisone in a water solution. The composition may be formulated in a vehicle or co-solvent. Although water itself may make up the entire vehicle, typical ear drop formulations may contain a co-solvent to assist in solubilization and of incorporation of water insoluble ingredients.

The vehicle may be a physiologically active ingredient that provides additional benefits such as solubilizing other active ingredients or acting as a preservative. Examples of physiologically active ingredients that are also water soluble include antimicrobial quaternary ammonium compounds such as cetrimide alkylaryltrialkylammonium chloride, alkylaryltrimethylammonium chloride, amantanium bromide, benzalkonium chloride, benzethonium chloride, benzododecinium bromide, cetalkonium chloride, cethexonium bromide, cetrimonium bromine, and cetyldimethylethylammonium bromide.

The vehicles may be viscosity building agents such as polyvinyl alcohol, polyvinyl pyrrolidone, methyl cellulose, hydroxy propyl methylcellulose, hydroxyethyl cellulose, carboxymethyl cellulose, hydroxy propyl cellulose or other agents know to those skilled in the art. The vehicle may be viscous to permit maximum contact time between the medication and the tissues of the ear.

The composition may include an additional vehicle that is a penetration enhancer. A penetration enhancer, also called a permeation enhancer, is an agent used to increase the permeability of the skin to a pharmacologically active agent to increase the rate at which the drug diffuses through the skin and enters the tissues and bloodstream. Examples of penetration enhancers include: alcohols, such as ethanol and isopropanol; polyols, such as n-alkanols, limonene, terpenes, dioxolane, propylene glycol, ethylene glycol, other glycols, and glycerol; sulfoxides, such as dimethylsulfoxide (DMSO), dimethylformamide, methyl dodecyl sulfoxide, dimethylacetamide; esters, such as isopropyl myristate/palmitate, ethyl acetate, butyl acetate, methyl proprionate, and capric/caprylic triglycerides; ketones; amides, such as acetamides; oleates, such as triolein; various surfactants, such as sodium lauryl sulfate; various alkanoic acids, such as caprylic acid; lactam compounds, such as azone; alkanols, such as oleyl alcohol; dialkylamino acetates, and admixtures thereof.

A number of patents disclose the use of penetration enhancers to deliver medications transdermally. For example, U.S. Pat. No. 5,837,289 discloses the use of two penetration enhancers in a cream to deliver an extensive list of medications, which is expressly incorporated by reference herein. In another example, a skin permeation enhancer composition is used comprising a lower aliphatic ester of a lower aliphatic carboxyl acid in combination with a lower alkanol to administer an active agent. See, for example, U.S. Pat. No. 5,238,933, which is expressly incorporated by reference herein. In another example, a vegetable oil-based skin permeation enhancer to deliver active agents through the skin has been used. See, for example, U.S. Pat. No. 5,229,130, which is expressly incorporated by reference herein.

Many ear drop formulations contain a topical anesthetic for immediate relief of the pain. However, due to negative or deleterious side effects, the composition may be free of analgesics or topical anesthetics. For example, the composition may be substantially free of the topical anesthetic pramoxine. Pramoxine is a nonsteroidal antipruritic that functions as a local anesthetic. Topical anesthetics are commonly added in ear infection treatment compositions due to its ability to numb the pain due to ailments such as otitis externa. However, there are numerous advantages to not including an analgesic or topical anesthetic, and specifically, a topical anesthetic such as pramoxine, in the compositions of the present invention. First, numbing the pain may mask symptoms of an advancing infection. Osguthorpe et al., supra. Hence, a topical anesthetic may hide symptoms that may cause serious if not permanent damage and be a risk that outweighs the quick relief it provides. Moreover, pramoxine is known to cause contact dermititis or induce inflammation to an allergic response. Hence, adding pramoxine or another topical anesthetic or analgesic may knock out or reduce the benefits of hydrocortisone or other added anti-inflammatory within the composition. For these reasons, a specific embodiment may be substantially free of pramoxine or other topical anesthetics.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

### EXAMPLES

Without requiring further elaboration, it is believed that one skilled in the art, using the preceding description, can utilize the present invention to the fullest extent. The following examples are illustrative only, and not limiting of the remainder of the disclosure in any way whatsoever.

### Example 1

A composition of the following formulation is prepared in an aqueous form, including the appropriate excipients, by standard methods known to those of ordinary skill in the art:
Hydrocortisone
   1.0%
Chloroxylenol
   0.1%
Benzalkonium Chloride
   0.01%
Propylene Glycol
   3.0%

### Example 2

A study is undertaken to evaluate the effectiveness of the compositions of the present invention in the treatment of patients. The objective of the study is to determine whether three drops in the patients ear three times a day of the combination of the compositions of the present invention results in a rapid improvement of the symptoms of otitis externa such as pain, itchiness and redness or swelling in the external auditory canal.

A double-blind, placebo controlled study is conducted over a 10 day period. A total of 120 subjects, all presenting for treatment of symptoms of otitis externa, are chosen for the study. The patients range in age from 14 to 50 years old.

An initial assessment of the symptoms of each patient is conducted when the patients initially present for treatment. The patient rates the severity of the symptoms on a 4-point scale (0: absent; 1: mild; 2: moderate; 3: severe). For inclusion in the study, a patient must be rated with a score of two or above for otitis externa.

The 120 subjects chosen for the study are separated into two separate groups of 60. The characteristics of the symptoms between the two groups are comparable. The first group is administered a three ear drop dose of the composition of the present invention at the onset of the symptoms of otitis externa where the severity has reached at least a score of 2 as scored by the patient. The second group is administered a placebo medication at the onset of the symptoms of otitis externa that is similar in all respects to the administered composition except for the exclusion of the active ingredients hydrocortisone, chloroxylenol and benzalkonium chloride. The various symptoms of otitis externa are evaluated by the patient every 24 hrs after the beginning of treatment of the study medication using the same 4-point scale. The symptoms evaluated are pain severity, itchiness and redness or swelling in the external auditory canal.

The assessment of the relief for pain severity, itchiness and redness or swelling in the external auditory canal is conducted for each subject group. The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 0.5, 1, 2, 3 and 4 hours, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

This study will demonstrate the efficacy of the composition of the present invention in treating the symptoms of otitis externa. Regarding potential adverse effects of taking the medication, if there are no significant differences between the two therapeutic groups, this study will demonstrate that the administration of the composition of the present invention is effective at treating symptoms of otitis externa in addition to being well-tolerated by the patients.

1. A composition comprising an antibacterial, an anti-inflammatory, and an anti-septic, wherein said composition is substantially free of pramoxine and said composition is for otic use.

2. The composition of claim 1, wherein said composition is substantially free of an anesthetic.

3. The composition of claim 1, wherein said antibacterial is selected from one or more of the group consisting of chloroxylenol (parachlorometaxylenol), acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin and zorbamycin;

4. The composition of claim 1, wherein said anti-inflammatory is selected from one or more of the group consisting of hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone prostaglandin H synthetase inhibitors (Cox I or Cox II), flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam, carprofen, cyclooxygenase type II selective inhibitors, vioxx, celecoxib, P54, etodolac, L-804600, S-33516; PAF antagonists, A-137491, ABT-299, apafant, bepafant, minopafant, E-6123, BN-50727, nupafant, modipafant, PDE IV inhibitors, ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast.

5. The composition of claim 1, wherein said anti-septic is selected from one or more of the group consisting of benzalkonium chloride, cetyl trimethylammonium bromide, cetylpyridinium chloride, cetylpyridinium chloride, benzethonium chloride, 2% aqueous iodine, boric acid, chlorhexidine gluconate, hydrogen peroxide, octenidine dihydrochloride, ethanol, isopropanol, n-propanol, phenylethyl alcohol, benzyl alcohol, and phenoxyethyl alcohol.

6. The composition of claim 1, wherein said antibacterial is chloroxylenol, said anti-inflammatory is hydrocortisone and said anti-septic is benzalkonium chloride, and wherein said composition is administrable to a patient.

7. The composition of claim 6, wherein said composition is in range amounts per 1 ml of solution.

8. The composition of claim, wherein said composition is substantially free of other added active ingredients.

9. The composition of claim 8, wherein said other active ingredient is another antibacterial.

10. The composition of claim 8, wherein said other active ingredient is another anti-inflammatory.

11. The composition of claim 8, wherein said other active ingredient is another anti-septic.

12. The composition of claim 9, wherein said antibacterial is selected from one or more of the group consisting of acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir, ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamicillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin and zorbamycin

13. The composition of claim 10, wherein said anti-inflammatory is selected from one of more of the group consisting of hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone prostaglandin H synthetase inhibitors (Cox I or Cox II), flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam, carprofen, cyclooxygenase type II selective inhibitors, vioxx, celecoxib, P54, etodolac, L-804600, S-33516; PAF antagonists, A-137491, ABT-299, apafant, bepafant, minopafant, E-6123, BN-50727, nupafant, modipafant, PDE IV inhibitors, ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast.

14. The composition of claim 11, wherein said anti-septic is selected from one or more of the group consisting of cetyl trimethylammonium bromide, cetylpyridinium chloride, cetylpyridinium chloride, benzethonium chloride, 2% aqueous iodine, boric acid, chlorhexidine gluconate, hydrogen peroxide, octenidine dihydrochloride, ethanol, isopropanol, n-propanol, phenylethyl alcohol, benzyl alcohol, and phenoxyethyl alcohol.

15. The composition of claim 7, wherein said chloroxylenol is present in the range of about 1.5 mg to about 0.5 mg.

16. The composition of claim 7, wherein said hydrocortisone is present in the range of about 15 mg to about 5 mg.

17. The composition of claim 7, wherein said benzalkonium chloride is present in the range of about 0.15 mg to about 0.05 mg.

18. The composition of claim 7, wherein said chloroxylenol is present in the range of about 1.25 mg to about 0.75 mg.

19. The composition of claim 7, wherein said hydrocortisone is present in the range of about 12.5 mg to about 7.5 mg.

20. The composition of claim 7, wherein said benzalkonium chloride is present in the range of about 0.125 mg to about 0.075 mg.

21. The composition of claim 7, wherein said chloroxylenol is present in the range of about 1.1 mg to about 0.9 mg.

22. The composition of claim 7, wherein said hydrocortisone is present in the range of about 11 mg to about 9 mg.

23. The composition of claim 7, wherein said benzalkonium chloride is present in the range of about 0.11 mg to about 0.09 mg.

24. The composition of claim 7, wherein said composition comprises about 1.25 mg to about 0.75 mg of chloroxylenol; about 12.5 mg to about 7.5 mg of hydrocortisone; about 0.125 mg to about 0.075 mg of benzalkonium chloride.

25. The composition of claim 7, wherein said composition comprises about 1.5 mg to about 0.5 mg of chloroxylenol; about 15 mg to about 5 mg of hydrocortisone; about 0.15 mg to about 0.05 mg of benzalkonium chloride.

26. The composition of claim 7, wherein said composition comprises about 1.1 mg to about 0.9 mg of chloroxylenol; about 11 mg to about 9 mg of hydrocortisone; about 0.11 mg to about 0.09 mg of benzalkonium chloride.

27. The composition of claim 7, wherein said chloroxylenol is present in the amount of about 1 mg.

28. The composition of claim 7, wherein said hydrocortisone is present in the amount of about 10 mg.

29. The composition of claim 7, wherein said benzalkonium chloride is present in the amount of about 0.1 mg.

30. The composition of claim 7, wherein said chloroxylenol is present in the amount of about 1 mg; said hydrocortisone is present in the amount of about 10 mg; and said benzalkonium chloride is present in the amount of about 0.1 mg.

31. The composition of claim 1, wherein said composition is formulated within a vehicle.

32. The composition of claim 31, wherein said vehicle is aqueous, non-aqueous, oleaginous and viscous.

33. The composition of claim 31, wherein said vehicle is selected from one or more of the group consisting of glycols, alcohols, ethanol, isopropanol, glycerol, sorbitol, 2-methoxy ethanol, mannitol, diethyl ether, dipropyl ether, methoxy polyoxyethylenes, polyoxyethylene glycerols, polyoxyethylene sorbitols, stearoyl diacetin glycerin, dehydrated glycerin, mineral oil, water, anhydrous glycerin, glycerol polyvinyl alcohol, polyvinyl pyrrolidone, methyl cellulose, hydroxy propyl methylcellulose, hydroxyethyl cellulose, carboxymethyl cellulose, hydroxy propyl cellulose sorbitol, xylitol, cetrimide alkylaryltrialkylammonium chloride, alkylaryltrimethylammonium chloride, amantanium bromide, benzethonium chloride, benzododecinium bromide, cetalkonium chloride, cethexonium bromide, cetrimonium bromine, and cetyldimethylethylammonium bromide.

34. The composition of claim 33, wherein said glycol is selected from one or more of the group consisting of polyethylene glycol (PEG) 300 or any other molecular weight of PEG, propylene glycol, propylene glycol diacetate, diethylene glycol, ethylene glycol, hexalene glycol and hexenyl glycol.

35. The composition of claim 34, wherein said propylene glycol is present in the concentration range of about 2 percent to 4 percent.

36. The composition of claim 34, wherein said propylene glycol is present in the concentration of about 3 percent.

37. The composition of claim 1, wherein said composition comprises a skin permeation enhancer.

38. The composition of claim 37, wherein said skin permeation enhancer is selected from one or more of the group consisting of ethanol, isopropanol, n-alkanols, limonene, terpenes, dioxolane, propylene glycol, ethylene glycol, glycerol, dimethylsulfoxide (DMSO), dimethylformamide, methyl dodecyl sulfoxide, dimethylacetamide, isopropyl myristate/palmitate, ethyl acetate, butyl acetate, methyl proprionate, capric/caprylic triglycerides; ketones, acetamides, triolein; sodium lauryl sulfate, alkanoic acids, caprylic acid, azone, oleyl alcohol and dialkylamino acetates.

39. The composition of claim 1, wherein said composition is administered to said patient to treat and/or alleviate the occurrence or negative effects of otic inflammation.

40. The composition of claim 1, wherein said composition is administered to said patient to treat and/or alleviate the occurrence or negative effects of otic infections.

41. The composition of claim 1, wherein said composition is administered to said patient to treat and/or alleviate the occurrence or negative effects of otitis externa.

42. The composition of claim 41, wherein said otitis externa is from one or more of the group consisting of acute otitis externa, chronic otitis externa.

43. The composition of claim 40, wherein said otic infections is from one or more of the group consisting of furunculosis, otomycosis, acute diffuse otitiss externa, acute localised otitis externa, chronic otitis externa, herpes oticus, dermatoses, malignant otitis externa, perichondritis, acute otitis media, serous otitis media, chronic otitis media, and vestibular neruonitis.

44. The composition of claim 1, wherein said composition is contained within a delivery system comprising a solution in a container that permits the administration of drops.

45. A method comprising administering to a patient the composition of claim 1.

46. A method comprising a patient taking the composition of claim 1.

47. The method of claim 45, wherein said composition is substantially free of an anesthetic.

48. The method of claim 45, wherein said antibacterial is selected from one or more of the group consisting of chloroxylenol (parachlorometaxylenol), acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenafe complex; chloramphenicol sodium succinate; chlorhexidine phosphanilate; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin and zorbamycin.

49. The method of claim 45, wherein said anti-inflammatory is selected from one or more of the group consisting hydrocortisone, hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone prostaglandin H synthetase inhibitors (Cox I or Cox II), flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam and carprofen, cyclooxygenase type II selective inhibitors, vioxx, celecoxib, P54, etodolac, L-804600, S-33516; PAF antagonists, A-137491, ABT-299, apafant, bepafant, minopafant, E-6123, BN-50727, nupafant and modipafant, PDE IV inhibitors, ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast.

50. The method of claim 45, wherein said anti-septic is selected from one or more of the group consisting of benzalkonium chloride, cetyl trimethylammonium bromide, cetylpyridinium chloride, cetylpyridinium chloride, benzethonium chloride, 2% aqueous iodine, boric acid, chlorhexidine gluconate, hydrogen peroxide, octenidine dihydrochloride, ethanol, isopropanol, n-propanol, phenylethyl alcohol, benzyl alcohol, and phenoxyethyl alcohol.

51. The method of claim 45, wherein said antibacterial is chloroxylenol, said anti-inflammatory is hydrocortisone and said anti-septic is benzalkonium, and wherein said composition is administrable to a patient.

52. The method of claim 51, wherein said composition is in range amounts per 1 ml of solution.

53. The method of claim 45, wherein said composition is substantially free of other added active ingredients.

54. The method of claim 53, wherein said other active ingredient is another antibacterial.

55. The method of claim 53, wherein said other active ingredient is another anti-inflammatory.

56. The method of claim 53, wherein said other active ingredient is another anti-septic.

57. The method of claim 54, wherein said antibacterial is selected from one or more of the group consisting of acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; ganciclovir and ganciclovir sodium; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin and oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillins such as penicillin g benzathine, penicillin g potassium, penicillin g procaine, penicillin g sodium, penicillin v, penicillin v benzathine, penicillin v hydrabamine, and penicillin v potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin b sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin and zorbamycin.

58. The method of claim 55, wherein said anti-inflammatory is selected from one or more of the group consisting hydroxyltriamcinolone alphamethyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclarolone acetonide, fludrocortisone, flumethasone pivalate, fluosinolone acetonide, fluocinonide, flucortine butylester, fluocortolone, fluprednidene (fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amc, amcinafide, betamethasone and the balance of its esters, chlorprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylproprionate, hydrocortamate, meprednisone, paramethasone, prednisolone, prednisone, beclomethasone dipropionate, betamethasone dipropionate, triamcinolone prostaglandin H synthetase inhibitors (Cox I or Cox II), flurbiprofen, ketorolac, suprofen, nepafenac, amfenac, indomethacin, naproxen, ibuprofen, bromfenac, ketoprofen, meclofenamate, piroxicam, sulindac, mefanamic acid, diflusinal, oxaprozin, tolmetin, fenoprofen, benoxaprofen, nabumetome, etodolac, phenylbutazone, aspirin, oxyphenbutazone, NCX-4016, HCT-1026, NCX-284, NCX-456, tenoxicam, carprofen, cyclooxygenase type II selective inhibitors, vioxx, celecoxib, P54, etodolac, L-804600, S-33516; PAF antagonists, A-137491, ABT-299, apafant, bepafant, minopafant, E-6123, BN-50727, nupafant and modipafant, PDE IV inhibitors, ariflo, torbafylline, rolipram, filaminast, piclamilast, cipamfylline, CG-1088, V-11294A, CT-2820, PD-168787, CP-293121, DWP-205297, CP-220629, SH-636, BAY-19-8004, and roflumilast.

59. The method of claim 56, wherein said anti-septic is selected from one or more of the group consisting of cetyl trimethylammonium bromide, cetylpyridinium chloride, cetylpyridinium chloride, benzethonium chloride, 2% aqueous iodine, boric acid, Chlorhexidine Gluconate, Hydrogen peroxide, Octenidine dihydrochloride, ethanol, isopropanol, n-propanol, phenylethyl alcohol, benzyl alcohol, and phenoxyethyl alcohol.

60. The method of claim 51, wherein said chloroxylenol is present in the range of about 1.5 to about 0.5 mg.

61. The method of claim 51, wherein said hydrocortisone is present in the range of about 15 mg to about 5 mg.

62. The method of claim 51, wherein said benzalkonium chloride is present in the range of about 0.15 mg to about 0.05 mg.

63. The method of claim 51, wherein said chloroxylenol is present in the range of about 1.25 to about 0.75 mg.

64. The method of claim 51, wherein said hydrocortisone is present in the range of about 12.5 mg to about 7.5 mg.

65. The method of claim 51, wherein said benzalkonium chloride is present in the range of about 0.125 mg to about 0.075 mg.

66. The method of claim 51, wherein said chloroxylenol is present in the range of about 1.1 mg to about 0.9 mg.

67. The method of claim 51, wherein said hydrocortisone is present in the range of about 11 mg to about 9 mg.

68. The method of claim 51, wherein said benzalkonium chloride is present in the range of about 0.11 mg to about 0.09 mg.

69. The method of claim 51, wherein said composition comprises about 1.25 mg to about 0.75 mg of chloroxylenol; about 12.5 mg to about 7.5 mg of hydrocortisone; about 0.125 mg to about 0.075 mg of benzalkonium chloride.

70. The method of claim 51, wherein said composition comprises about 1.5 mg to about 0.5 mg of chloroxylenol; about 15 mg to about 5 mg of hydrocortisone; about 0.15 mg to about 0.05 mg of benzalkonium chloride.

71. The method of claim 51, wherein said composition comprises about 1.1 mg to about 0.9 mg of chloroxylenol; about 11 mg to about 9 mg of hydrocortisone; about 0.11 mg to about 0.09 mg of benzalkonium chloride.

72. The method of claim 51, wherein said chloroxylenol is present in the amount of about 1 mg.

73. The method of claim 51, wherein said hydrocortisone is present in the amount of about 10 mg.

74. The method of claim 51, wherein said benzalkonium chloride is present in the amount of about 0.1 mg.

75. The method of claim 45, wherein said antibacterial, anti-inflammatory an anti-septic are formulated into a vehicle.

76. The method of claim 75, wherein said vehicle is aqueous, non-aqueous, oleaginous or viscous.

77. The method of claim 76, wherein said vehicle is selected from one or more of the group consisting of glycols, alcohols, ethanol, isopropanol, glycerol, sorbitol, 2-methoxy ethanol, mannitol, diethyl ether, dipropyl ether, methoxy polyoxyethylenes, polyoxyethylene glycerols, polyoxyethylene sorbitols, stearoyl diacetin glycerin, dehydrated glycerin, mineral oil, water, anhydrous glycerin, glycerol polyvinyl alcohol, polyvinyl pyrrolidone, methyl cellulose, hydroxy propyl methyl cellulose, hydroxyethyl cellulose, carboxymethyl cellulose, hydroxy propyl cellulose sorbitol, xylitol, cetrimide alkylaryltrialkylammonium chloride, alkylaryltrimethylammonium chloride, amantanium bromide, benzethonium chloride, benzododecinium bromide, cetalkonium chloride, cethexonium bromide, cetrimonium bromine, and cetyldimethylethylammonium bromide.

78. The method of claim 77, wherein said glycol is selected from one or more of the group consisting of polyethylene glycol (PEG) 300 or any other molecular weight of PEG, propylene glycol, propylene glycol diacetate, diethylene glycol, ethylene glycol, hexalene glycol and hexenyl glycol.

79. The method of claim 78, wherein said propylene glycol is present in the concentration range of about 2 percent to 4 percent.

80. The method of claim 78, wherein said propylene glycol is present in the concentration of about 3 percent.

81. The method of claim 45, wherein said composition comprises a skin permeation enhancer.

82. The method of claim 81, wherein said skin permeation enhancer is selected from one or more of the group consisting of ethanol, isopropanol, n-alkanols, limonene, terpenes, dioxolane, propylene glycol, ethylene glycol, glycerol, dimethylsulfoxide (DMSO), dimethylformamide, methyl dodecyl sulfoxide, dimethylacetamide, isopropyl myristate/palmitate, ethyl acetate, butyl acetate, methyl proprionate, capric/caprylic triglycerides; ketones, acetamides, triolein; sodium lauryl sulfate, alkanoic acids, caprylic acid, azone, oleyl alcohol and dialkylamino acetates.

83. The method of claim 45, wherein said composition is administered to said patient to treat and/or alleviate the occurrence or negative effects of otic inflammation.

84. The method of claim 45, wherein said composition is administered to said patient to treat and/or alleviate the occurrence or negative effects of otic infections.

85. The method of claim 45, wherein said composition is administered to said patient to treat and/or alleviate the occurrence or negative effects of otitis externa.

86. The method of claim 85, wherein said otitis externa is from one or more of the group consisting of acute otitis externa, chronic otitis externa.

87. The method of claim 84, wherein said otic infections is from one or more of the group consisting of furunculosis, otomycosis, acute diffuse otitis externa, acute localised otitis externa, chronic otitis externa, herpes oticus, dermatoses, malignant otitis externa, perichondritis, acute otitis media, serous otitis media, chronic otitis media, and vestibular neruonitis.

88. The method of claim 45, wherein said composition is contained within a delivery system comprising a solution in a container that permits the administration of drops.

89. The method of claim 45, wherein said composition is administered to said patient at a frequency selected from the group consisting of once a day, twice a day, three times a day, four times a day, five times a day, six times a day, seven times a day, eight times a day, nine times a day, ten times a day, eleven times a day and twelve times a day.

90. The method of claim 46, wherein said patient takes said composition at a frequency selected from the group consisting of once a day, twice a day, three times a day, four times a day, five times a day, six times a day, seven times a day, eight times a day, nine times a day, ten times a day, eleven times a day and twelve times a day.

91. The method of claim 45, wherein said composition is administered to said patient at a frequency selected from the group consisting of once every hour, once every two hours, once every three hours, once every four hours, once every five hours, once every six hours, once every seven hours, once every eight hours, once every nine hours, once every ten hours, once every eleven hours, once every twelve hours, once every thirteen hours, once every fourteen hours, once every fifteen hours, once every sixteen hours, once every seventeen hours, once every eighteen hours, once every nineteen hours, once every twenty hours, once every twenty one hours, once every twenty two hours, once every twenty three hours and once every twenty four hours.

92. The method of claim 46, wherein said patient takes said composition at a frequency selected from the group consisting of once every hour, once every two hours, once every three hours, once every four hours, once every five hours, once every six hours, once every seven hours, once every eight hours, once every nine hours, once every ten hours, once every eleven hours, once every twelve hours, once every thirteen hours, once every fourteen hours, once every fifteen hours, once every sixteen hours, once every seventeen hours, once every eighteen hours, once every nineteen hours, once every twenty hours, once every twenty one hours, once every twenty two hours, once every twenty three hours and once every twenty four hours.

93. The method of claim 45, wherein said composition is administered to said patient in a dose selected from the group consisting of 1 drop, 2 drops, 3 drops, 4 drops, 5 drops, 6 drops, 7 drops and 8 drops.

94. A composition consisting of chloroxylenol, hydrocortisone, benzalkonium chloride and one or more excipients.

95. The composition of claim 94, wherein said composition has about 1.5 mg to about 0.5 mg chloroxylenol per 1 ml of solution, about 15 mg to about 5 mg hydrocortisone per 1 ml of solution, and about 0.15 mg to about 0.05 mg benzalkonium chloride per 1 ml of solution.

96. The composition of claim 95, wherein said composition has about 1.0 mg chloroxylenol per 1 ml of solution, about 10 mg hydrocortisone per 1 ml of solution, and about 0.10 mg benzalkonium chloride per 1 ml of solution.

97. A method consisting of administering to a patient the composition of claim 94.

98. The method of claim 97, wherein said composition has about 1.5 mg to about 0.5 mg chloroxylenol per 1 ml of solution, about 15 mg to about 5 mg hydrocortisone per 1 ml of solution, and about 0.15 mg to about 0.05 mg benzalkonium chloride per 1 ml of solution.

99. The method of claim 98, wherein said composition has about 1.0 mg chloroxylenol per 1 ml of solution, about 10 mg hydrocortisone per 1 ml of solution, and about 0.10 mg benzalkonium chloride per 1 ml of solution.
